(19) United States
(12) Patent Application Publication
(10) Pub. No.: US 2007/0162004 A1
(43) Pub. Date: Jul. 12, 2007

(54) Device for the lateral stabilization of the spine

(76) Inventors: Alain Tornier, Saint Ismier (FR); Irene Ferrari Gosset, Saint Vincent De Mercuze (FR)

Correspondence Address: Ralph A. Dowell of DOWELL & DOWELL P.C., 2111 Eisenhower Ave, Suite 406, Alexandria, VA, 22314, US

(21) Appl. No.: 11/634,378

(22) Filed: Dec. 6, 2006

Related U.S. Application Data
(60) Provisional application No. 60/748,605, filed on Dec. 9, 2005.

(30) Foreign Application Priority Data
Dec. 7, 2005 (FR) 05 12426

(51) Int. Cl. A61F 2/30

(52) U.S. Cl. 606/61

(57) ABSTRACT

The device (4) comprises two vertebral elements (10, 20) each adapted to be fixed to the same lateral side of the body of at least two adjacent vertebrae (1, 2). In order to guide the vertebrae effectively and stably in order to reproduce an articulating intervertebral joint, the elements delimit respective surfaces (14A, 26A) for the relative guiding of the two elements, which surfaces are so adapted that, when the elements are implanted on the corresponding vertebrae, they cooperate with one another by complementarity of forms, in such a manner that they define an axis of rotation (X-X) about which the two elements are able to tilt one relative to the other and which extends both according to a direction substantially mediolateral to the spine and in the intervertebral disk space (7) separating the two adjacent vertebrae.

The present invention relates to a device for the lateral stabilization of the spine, which device is to be implanted along the vertebral column in the region of one or both of its left and right lateral sides, in order to stabilize at least two vertebrae one relative to the other. Such dynamic stabilization is desirable especially within the context of the treatment of degenerative or traumatized spine. The invention relates more particularly to the treatment of the dorsolumbar spine, but is likewise applicable to the treatment of the cervical spine.

For the treatment of an intervertebral instability, a first known possibility comprises fusing two adjacent vertebrae, which amounts to depriving those two vertebrae of their relative freedom of movement. Totally rigid assemblies are implanted for that purpose in a fixed manner along the spine in order permanently to block the articulating joint between the two vertebrae to be fused. U.S. Pat. No. 6,296,644 accordingly proposes a vertebral assembly constituted by a plurality of vertebral elements which are to be fixed to the same number of vertebrae and which are connected in pairs by “lockable” joints: when the assembly is being fitted, the joints are movable in order to facilitate the relative positioning of the vertebral elements along the spine, and then, when fitting is complete, the joints are fixed permanently by means of rings having shape memory so that, in use, the vertebral elements are completely fixed relative to one another. However, this type of operation of arthrodesis of the vertebrae leads to degeneration of the adjacent disks, on which it is subsequently necessary to operate.

Another known possibility for treatment of the spine comprises operating at an earlier stage than that which involves arthrodesis. A first solution of that type is proposed in DE-U-298 14 320: a plurality of separate vertebral elements, each fixed to adjacent vertebrae, are in use movable relative to one another, while being connected in pairs by rectilinear telescopic joints according to the longitudinal direction of the spine. This movable assembly adapts to a certain development of the kinematic behavior of the spine, for example as it grows, but does not provide actual dynamic stabilization of the vertebrae and accordingly does not prevent, for example, crushing or deformation of the intervertebral disks.

A second solution is proposed in JP-A-10 277070: the posterior sides of two adjacent vertebrae are connected vertically by two pistons, a sleeve of resilient material being inserted between the male part and the female part of each piston. The transverse cross-section of each piston is elliptical, which centres the articulating movements between the male and female parts of each piston either in the piston or in a plane that passes through the two pistons, that is to say well behind the vertebrae. The kinematics imposed on the vertebrae is accordingly very different from the normal anatomical behavior of the spine, with considerable risks that the intervertebral disk will be pinched, or even crushed, at least in its anterior portion.

Other solutions aim to implant a lateral device for dynamic stabilization, such as those proposed in U.S. Pat. No. 5,423,816, U.S. Pat. No. 5,704,936 and U.S. Pat. No. 6,616,669. To that end, this type of device comprises, on the one hand, rigid elements that are to be anchored in the bone of the same lateral side of two adjacent vertebrae and, on the other hand, flexible joining elements between the rigid elements. These flexible elements, such as springs or flexible arms, extend laterally along the spine and thus relieve the intervertebral disk by reducing any excess pressure in the region of the articulating surfaces between that disk and the vertebrae. Such devices are more comfortable for the patient because they allow the mobility of the spine to be retained. However, the use of that type of dynamic device is found to be difficult in practice. Dimensioning of the flexibility of the joining elements is difficult because it must be adapted to each patient according to his pathology and morphology and, in the long term, the resilient behavior of those elements changes. If those parameters are poorly controlled, it is not possible to ensure that a kinematics appropriate to the spine is respected, which can lead to poor stabilization of the intervertebral spacing and to aggravation of the damage that it is desired to treat.

The object of the present invention is to propose a device for the lateral stabilization of the spine that reproduces the anatomical movements of the vertebrae more faithfully, is more effective for stabilizing the vertebrae to be treated and is more reliable over time.

To that end, the invention relates to a device for the lateral stabilization of the spine which is intended, in use, to reproduce an articulating intervertebral joint, comprising at least two vertebral elements which are each adapted to be fixed to the same lateral side of the body of at least two adjacent vertebrae, characterized in that the two vertebral elements delimit respective surfaces for the relative guiding of the two elements, which surfaces are so adapted that, when the elements are implanted on their corresponding vertebra, they cooperate one with the other by complementarity of forms, in such a manner that the surfaces define an axis of rotation about which the two elements are able to tilt one relative to the other and which extends both according to a direction substantially mediolateral to the spine and in the intervertebral disk space separating the two adjacent vertebrae.

The fact that the two vertebral elements are guided one relative to the other by surfaces that are carried by those elements and that cooperate by complementarity of forms makes it possible to confer on the device precise kinematic behavior that is stable over time. The imposed kinematics, namely a relative tilting between the two assemblies about a mediolateral or approximately mediolateral axis, ensures that the intervertebral articulating movements induced by stress on the spine, especially by bending/stretching, are efficiently centred about a precise axis whose predetermined position in the disk space is provided so that that behavior is quasi-identical with, or at least as similar as possible to, the normal anatomical behavior of the spine. In that manner, the cooperation of those guide surfaces resting one against the other can allow a satisfactory intervertebral spacing to be retained, while maintaining a predetermined vertical spacing of the vertebrae. The device according to the invention accordingly bears the majority, or even the totality, of the stresses applied to the intervertebral disk, which remains mobile. Furthermore, implantation of the device according to the invention is found to be particularly simple because the mobilities particular to the device reside substantially, or even exclusively, in the region of the guide surfaces carried by the two vertebral elements, the anchoring positions of which in the two vertebrae to be treated are chosen and fixed by the surgeon.

According to other advantageous features of this device, taken in isolation or according to all the technically possible combinations:
the axis of rotation extends in the intervertebral disk space that separates the two vertebrae equipped with the two vertebral elements;
the guide surfaces define a permanent axis of rotation when the vertebrae equipped with the two vertebral elements are displaced one relative to the other according to a bending/stretching movement of the spine;
the guide surfaces define a plurality of instantaneous axes of rotation when the vertebrae equipped with the two vertebral elements are displaced one relative to the other according to a bending/stretching movement of the spine;
the guide surfaces are likewise adapted to maintain a minimum spacing between the vertebrae according to the longitudinal direction of the spine;
the resultant of contact of the two guide surfaces extends substantially parallel to the longitudinal direction of the spine;
the guide surfaces are formed by a male part of one of the two vertebral elements and by a female part of the other vertebral element, the female part receiving the male part when the elements are implanted on their corresponding vertebra;
the female part delimits a receiver for receiving the male part, the base of the receiver carrying, at least partly, the corresponding guide surface;
the female part includes a medial wall and/or a lateral wall for delimiting the receiver, which walls are so adapted that, when the elements are implanted on their corresponding vertebra, they retain the male part according to a mediolateral direction;
the axis of rotation is a geometric axis;
the axis of rotation is defined by a rod which is capable of being inclined relative to a direction mediolateral to the spine, with a maximum angle of less than approximately 10°, the guide surfaces corresponding substantially to portions of a sphere whose centre is located on the axis of rotation;
the device comprises four vertebral elements associated in pairs that are provided for fixing to the left and right sides of the two vertebrae.

The invention will better be understood upon reading the following description, which is given solely by way of example and with reference to the drawings, in which:

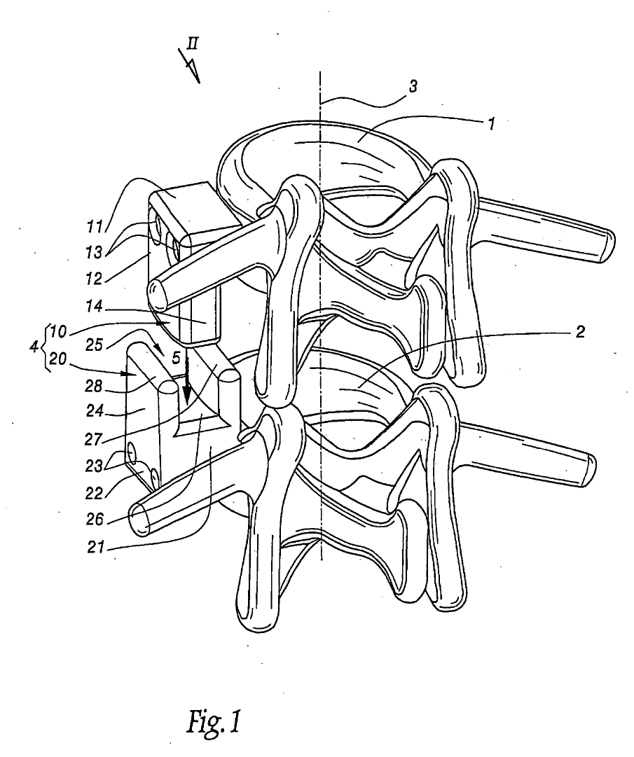

FIG. 1 is a view, in perspective, of two adjacent vertebrae equipped with a lateral stabilization device according to the invention, the vertebrae and the device being viewed from the rear and in a manner offset relative to the sagittal plane of the spine and being shown with an intervertebral spacing greater than normal for reasons of visibility;

FIG. 2 is a front view, according to arrow II in FIG. 1, of the lateral halves of the vertebrae and of the device, FIG. 2 accordingly corresponding to a view through the front of the spine;

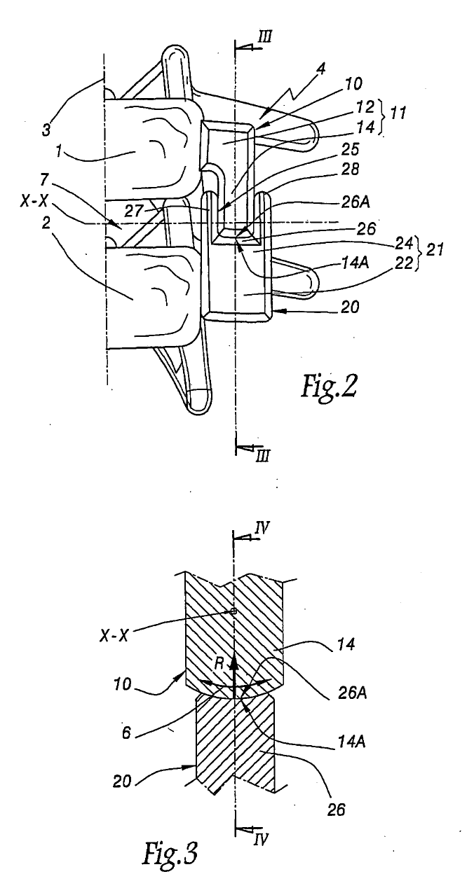

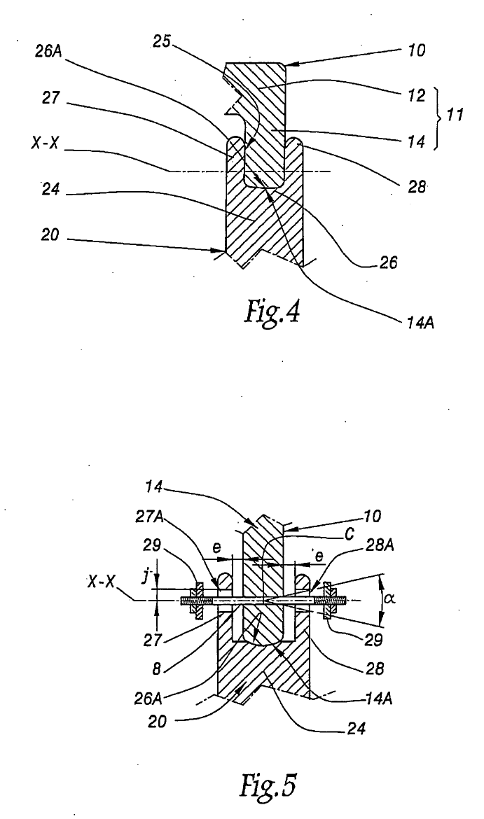

FIGS. 3 and 4 are sections according to lines III-III in FIG. 2 and IV-IV in FIG. 3, respectively; and

FIG. 5 is a section analogous to FIG. 4 of a variant of the device according to the invention.

FIGS. 1 and 2 show two adjacent vertebrae 1 and 2 of a lumbar spine of a human being. The longitudinal direction of the spine bears the reference numeral 3, the vertebrae 1 and 2 being separated one from the other, according to that direction, by an intervertebral disk, which is not shown in the figures for reasons of visibility. For convenience, the remainder of the description is oriented relative to the vertebrae in their anatomical position, that is to say the terms “posterior” or “rear”, “anterior” or “front”, “right”, “left”, “upper”, “lower”, etc. are to be understood relative to the spine of the patient standing upright. Likewise, the term “sagittal” corresponds to a direction in the anteroposterior direction, vertically on the median line of the spine, while the term “medial” corresponds to a direction substantially perpendicular to the sagittal plane of the spine, directed towards the spine, the term “lateral” corresponding to the opposite direction.

FIGS. 1 to 4 show a device 4 for the dynamic stabilization of the vertebrae 1 and 2, which device is implanted on the left side of the vertebrae in order to reproduce the articulating joint between the vertebrae, especially when the vertebrae are made to bend/stretch, while providing satisfactory intervertebral spacing. The device comprises an upper vertebral element 10 implanted in the region of the vertebra 1 and a lower vertebral assembly 20 implanted in the region of the vertebra 2.

Each vertebral element 10, 20 has a rigid body 11, 21 in a single piece, for example made of metal, which is adapted to be fixed to the left side of the vertebra 1, 2. To that end, apertures 13, 23 pass right through the upper part 12 of the body 11 and the lower part 22 of the body 21, according to a mediolateral direction, which apertures 13, 23 are intended to receive screws (not shown) for bone anchoring in the vertebral body of the vertebrae 1 and 2 in order firmly to immobilize the vertebral elements 10 and 20 relative to the vertebrae.

The lower part 14 of the element 10 and the upper part 24 of the element 20 are adapted to cooperate one with the other when the device 4 is implanted, as shown in FIG. 2. In frontal section, as in FIG. 4, that is to say in a substantially vertical cutting plane parallel to a mediolateral direction, the body 11, constituted by the upper and lower parts 12 and 14, which are integral with one another, has a cross-section in the form of an inverted L, while the upper part 24 has a generally U-shaped cross-section. The upper part 24 accordingly defines a receiver 25 which opens freely to the top and is intended to receive the lower part 14. The receiver 25 is delimited at the bottom by a bottom wall 26 and, at its sides, by a medial wall 27 and a lateral wall 28 which are parallel to one another, the walls 26, 27 and 28 being integral with the remainder of the body 21.

The mediolateral spacing between the walls 27 and 28 is substantially equal to the mediolateral dimension of the lower part 14 of the element 10 so that, when that part is received in the receiver 25, as indicated by arrow 5 in FIG. 1, it is retained according to a mediolateral direction, without any possibility of clearance, apart from functional play, as shown in FIGS. 2 and 4.

Unlike the surfaces facing the medial wall 27 and the lateral wall 28, the upper surface 26A of the bottom wall 26 is not flat but is curved, with its concavity turned upwards. More precisely, the surface 26A corresponds to a portion of a cylinder of axis X-X which extends above the bottom wall 26 and according to a substantially mediolateral direction, as shown in FIGS. 2 to 4. The surface 26A is adapted to guide the relative movements between the upper and lower elements 10 and 20 of the device 4, by cooperating by complementarity of forms with the lower end surface 14A of the part 14 of the element 10, that surface 14A also corresponding to a portion of a cylinder whose axis is substantially coincident with X-X when the elements are implanted. In other words, when the elements 10 and 20 are fixed to the vertebrae 1 and 2, the surfaces 14A and 26A rest one against the other in a tilting manner about the axis X-X, as indicated by arrow 6 in FIG. 3.

In order not to impede the relative tilting movements between the elements 10 and 20, the-anterior and posterior sides of the receiver 25 open freely to the outside.

In use, when the device 4 is implanted on the vertebrae 1 and 2, the surfaces 14A and 26A cooperate in such a manner as to guide the tilting between the vertebrae about the axis X-X. Since that axis advantageously extends in the intervertebral space 7 separating the vertebrae 1 and 2 according to the longitudinal direction 3 of the spine, in particular in the median region of that space, the tilting movements imposed by the cooperation of those surfaces are identical with, or at least very similar to, the anatomical intervertebral articulating movements generated when the spine is bent or stretched.

Lateral implantation of the device 4 is particularly simple and rapid because only the elements 10 and 20 are to be attached firmly to the vertebrae 1 and 2, by means of the above-mentioned bone anchorage screws. In practice, the elements 10 and 20 are placed in position simultaneously, with the lower part 14 of the element 10 received in the upper receiver 25 of the element 20, as indicated by arrow 5 in FIG. 1. In that manner, the surgeon is able to implant the device 4 with the surfaces 14A and 26A in contact with one another and for a predetermined extension configuration of the vertebrae. The device 4 thus maintains the longitudinal spacing of the vertebrae 1 and 2, according to the direction 3, under a constraint predetermined by the surgeon. It will be understood that, to that end, the surfaces 14A and 26A must extend generally according to an anteroposterior direction and thus cooperate in a pressing manner in the longitudinal direction 3 of the spine. In other words, the surfaces 14A and 26A cooperate one with the other to form a resultant of contact R which extends substantially parallel to that direction 3.

FIG. 5 shows a variant of the device 4 which is intended to allow the device to have slight freedom of internal clearances according to directions transverse to a strict mediolateral direction, providing greater comfort to the patient during twisting movements of the spine, that is to say rotary movements about the longitudinal direction of the spine, and/or movements of lateral inclination of the spine. To that end, this variant differs from the device of FIGS. 1 to 4 by the geometry of its cooperating guide surfaces 14A and 26A: in this variant, those surfaces correspond substantially to the same sphere portion centred on a point C located on a mediolateral axis X-X. In principle, the elements 10 and 20 are thus articulated one relative to the other in the manner of a ball-and-socket joint of centre C. In practice, only the tilting movement about the axis X-X is freely possible, the other permitted movements being limited by a rod 8 whose longitudinal axis corresponds to the axis X-X, which passes freely right through the lower part 14 of the element 10. The two longitudinal ends of the rod 8 are received, with a clearance j, in cylindrical apertures 27A and 28A, having a circular base, formed through the median wall 27 and the lateral wall 28 of the element 20. The diameter of the ends of the rod 8 is smaller than the diameter of the apertures 27A and 28A in order to define the clearance j. In that manner, rotary movements about the point C between the elements 10 and 20, other than the tilting movement about the axis X-X, are only permitted until the ends of the rod 8 abut one of the walls defining the apertures 27A and 28A.

For reasons of mechanical security, washers 30 are fitted around each end of the rod 8, which washers abut the medial side of the wall 27 and the lateral side of the wall 28. Each washer is associated with a locking screw 32, for example screwed round the threaded free end of the corresponding end of the rod 8.

In order to prevent the medial wall 27 and the lateral wall 28 from hindering those rotary clearance movements between the elements 10 and 20, a non-zero mediolateral spacing e is provided on the one hand between the wall 27 and the medial face of the part 14 and on the other hand between the wall 28 and the lateral face of the part 14. Furthermore, the retaining washers 29 mounted on the rod 8 are arranged at a distance from the walls 27 and 28.

By way of variation, instead of being arranged at a distance from the walls 27 and 28, the washers 29 may be in a portion of a sphere centred on the centre C, the outside faces of the walls 27 and 28 having the same geometry.

It will be noted that, unlike the device of FIGS. 1 to 4, in which the mediolateral tilting axis X-X is defined exclusively by the cooperation of the surfaces 14A and 26A, the tilting axis X-X between the elements 10 and 20 of the variant of the device of FIG. 5 is defined both by the cooperation of the spherical surfaces 14A and 26A and by the presence of the rod 8 that defines that axis. In practice, the direction of the tilting axis X-X for the device of FIGS. 1 to 4 is imposed in a manner substantially coincident with a mediolateral direction with respect to the spine, whereas, with the device of FIG. 5, that tilting axis X-X can, when the spine is stressed, be inclined relative to the direction mediolateral to the spine, the maximum angle a of that inclination being limited, however, to only several degrees, especially to about 10 degrees.

Various arrangements and variants of the device 4 described above can further be envisaged:
in the exemplary embodiment described in detail above, the tilting axis X-X between the elements 10 and 20 is a geometric axis; by way of variation, it is possible to provide a physical axis that extends through the receiver 25 and connects the medial wall 27 and the lateral wall 28, while being received in a complementary aperture that passes right through the lower part 14 of the element 10, according to a mediolateral direction;
the curvatures of the guide surfaces 14A and 26A can be reversed;
the male/female structure of the device 4 can be reversed so that, by way of variation, the upper vertebral element defines a lower receiver, analogous to the receiver 25, inside which the complementary upper part of the lower vertebral element is received;
rather than providing the strictly cylindrical surfaces 14A and 26A, so that they tilt by sliding one against the other about the only axis X-X, those surfaces can exhibit curved profiles which, during the relative tilting of the elements 10 and 20, define a plurality of instantaneous axes of rotation which are parallel to one another and extend according to mediolateral directions; and/or
in the exemplary embodiment shown in the Figures, the device 4 is implanted only in the region of the left side of the vertebrae 1 and 2; by way of variation, the device comprises, to replace the elements 10 and 20, two vertebral elements which are analogous to the elements 10 and 20 and are adapted to be implanted on the right side of the vertebrae; likewise, the device according to the invention can comprise four vertebral elements associated in pairs that are provided on the left and right sides of the vertebrae; in that case, in order to homogenize the kinematic behaviors of those two devices, it is possible to provide for a physical axis to connect the two pairs of elements, that axis extending according to the direction X-X and constituting a common tilting axis for the two pairs of elements, by passing through the intervertebral disk according to a mediolateral direction.

1. Device for the lateral stabilization of the spine which is intended, in use, to reproduce an articulating intervertebral joint, comprising at least two vertebral elements each adapted to be fixed to the same lateral side of the body of at least two adjacent vertebrae, wherein the two vertebral elements delimit respective surfaces for the relative guiding of the two elements, which surfaces are so adapted that, when the elements are implanted on their corresponding vertebra, they cooperate one with the other by complementarity of forms in such a manner that the surfaces define an axis of rotation about which the two elements can tilt one relative to the other and which extends both according to a direction substantially mediolateral to the spine and in the intervertebral disk space separating the two adjacent vertebrae.

2. Device according to claim 1, wherein the guide surfaces define a permanent axis of rotation when the vertebrae equipped with the vertebral elements are displaced one relative to the other according to a bending/stretching movement of the spine.

3. Device according to claim 1, wherein the guide surfaces define a plurality of instantaneous axes of rotation when the vertebrae equipped with the two vertebral elements are displaced one relative to the other according to a bending/stretching movement of the spine.

4. Device according to claim 1, wherein the guide surfaces are also adapted to maintain a minimum spacing between the vertebrae according to the longitudinal direction of the spine.

5. Device according to claim 4, wherein the resultant of contact of the two guide surfaces extends substantially parallel to the longitudinal direction of the spine.

6. Device according to any claim 1, wherein the guide surfaces are formed by a male part of one of the two vertebral elements and by a female part of the other vertebral element, the female part receiving the male part when the elements are implanted on their corresponding vertebra.

7. Device according to claim 6, wherein the female part delimits a receiver for receiving the male part, the base of the receiver carrying, at least partly, the corresponding guide surface.

8. Device according to claim 7, wherein the female part includes a medial wall and/or a lateral wall for delimiting the receiver, which walls are so adapted that, when the elements are implanted on their corresponding vertebra, they retain the male part according to a mediolateral direction.

9. Device according to claim 1, wherein said axis of rotation is a geometric axis.

10. Device according to claim 1, wherein the axis of rotation is defined by a rod capable of being inclined relative to a direction mediolateral to the spine, with a maximum angle of less than approximately 10°, the guide surfaces corresponding substantially to portions of a sphere whose centre is located on the axis of rotation.

11. Device according to claim 1, wherein it comprises four vertebral elements associated in pairs that are provided to be fixed to the left and right sides of the two vertebrae.
